(19) United States
(12) Patent Application Publication
(10) Pub. No.: US 2021/0379323 A1
(43) Pub. Date: Dec. 9, 2021

(54) AUTOMATIC OXYGEN THERAPY DEVICE

(71) Applicants: Liji Huang, Santa Clara, CA (US); Chih-Chang Chen, Cupertino, CA (US)

(72) Inventors: Liji Huang, Santa Clara, CA (US); Chih-Chang Chen, Cupertino, CA (US)

(73) Assignee: Siargo Ltd., Santa Clara, CA (US)

(21) Appl. No.: 16/896,643

(22) Filed: Jun. 9, 2020

(51) Int. Cl. A61M 16/16; A61M 16/00; A61M 16/10; A61M 16/20; G16H 40/67; G16H 40/63

(52) U.S. Cl. CPC A61M 16/162; A61M 2202/206; A61M 16/1005; A61M 16/203; G16H 40/67; G16H 40/63; A61M 2016/003; A61M 2016/1025; A61M 2016/0027; A61M 2205/8206; A61M 2205/0244; A61M 2205/3553; A61M 2205/3368; A61M 2205/3334; A61M 2205/18; A61M 16/0003

(57) ABSTRACT

The design and structure of a fully automatic oxygen therapy apparatus is exhibited in this disclosure. The apparatus integrates a MEMS mass flow meter, an oximeter, a proportional valve and a smart liquid bottle. The control unit of the apparatus is embedded with a wireless communication device and powered by a battery pack. This apparatus is designed to replace the mechanical oxygen rotameter used in today's hospital or homecare oxygen therapy applications. With a set recipe or parameters locally or remotely, the disclosed apparatus can perform a fully automatic oxygen therapy for recovering the blood oxygen level of patient, without the frequent attention of the therapy administrator, and especially it significantly reduces the possibility of cross infection to the administrator during the attendance of the oxygen therapy process. The therapy process data are relayed to local users as well as a designated cloud or data center. This disclosure will be beneficial for both medical staffs and patient.

### BACKGROUND OF THE INVENTION

### 1. Field of the Invention

This invention generally relates to gas flow measurement, and it particularly relates to an oxygen therapy apparatus that provides automatic oxygen delivery at a mass flow rate and consumption with feedback by the human saturated oxygen level or concentration in hospital and home care applications. This invention is further related to medical devices with digital data process and automation.

### 2. Description of the Related Art

Patients with respiratory diseases are often required oxygen therapy for boosting the peripheral capillary oxygen saturation. For over a century, such an oxygen treatment is realized by administrating the oxygen via titration of the flow of the supplied oxygen via a pure mechanical controlled mechanism. The whole oxygen therapy system is a simple oxygen rotameter and an attached water bottle (W. Haumann and C. P. Mulcahey, Oxygen therapy system, U.S. Pat. No. 3,199,303, Aug. 10, 1965). The rotameter is used to control the amount of oxygen to be given to the patient. The oxygen will then go through a humidification process via the attached water bottle attached to the rotameter. The rotameter is a mechanical flow meter having a low accuracy and it metering capability is often deviated with the changes of the oxygen delivery pressure and temperature. In addition, this therapy system can only provide a fixed and uncontrolled flow to the patient and has no connection to the actual function of boosting the patient's blood oxygen level or concentration which is measured by the independent oximeter. The therapy administrator who is often performed by a train nurse has to visit the patient multiple times to perform the necessary measurement and ensure the patient's blood oxygen level is moving to the right direction. The nurse also needs to adjust manually the oxygen flow rate for the effective therapy. In a few of recent publications (Davidson J. et al., Precision and accuracy of oxygen flow meters used at hospital settings, Respiratory Care, V. 57, 1071 (2012)), it was found that a vast of the current oxygen flow meters were erratic, leading to the difficulties for an accuracy judgement for the correct amount of the oxygen that should be used for boost the saturated blood oxygen level of the patients under treatment. By no means the nurse and even experienced medical doctors will know precisely the necessary time and dosage that a particular patient should take for re-generating the normal saturated blood oxygen level. It will then solely depend on the manual measurements experiences by the nurse to make the next guess as for whether or not to increase the oxygen dosage. The labor-intensive tasks and inaccurate process control of the oxygen therapy are particularly very much undesirable in the cases if the respiratory disease is infectious, such as the current COVID-19 pandemic situation when the oxygen therapy has been widely used to assist the recovery of the patients with light to heavy symptoms. The frequent visit by the nurses will increase the risk of cross-infections, and the inaccuracy of the oxygen dosage will also risk the patient's chance of timely recovery, or some reports indicated that the improperly usage of the oxygen therapy may create adverse effects to life threatening results in particular for the COVID-19 cases.

There are many disclosures related to the improvement of the oxygen therapy delivery approaches, such as improvement of the valve (R. D. Vann and S. R. Muza, Method and apparatus for supplemental oxygen delivery, U.S. Pat. No. 6,192,884, Feb. 27, 2001), addition of flow monitoring device (S. E. Culton, Warning device for oxygen delivery system failure, U.S. Pat. No. 6,386,196, May 14, 2002), addition of the monitoring of the blood oxygen content level (M. F. Schmidt, J. S. Buan and C. A. Nordman, Control of supplemental respiratory oxygen, U.S. Pat. No. 6,561,187, May 13, 2003) and addition of automation (T. Tyomkin and E. Tyomkin, Automatically regulating oxygen flow to a patient, U.S. Pat. No. 6,675,798, Jan. 13, 2004). However, disclosures for the improvement of the oxygen meters are hardly found even the meter is the critical device for the titration of the oxygen administration to patient. For example, the disclosure for the automated control by Tyomkins is only a conceptual illustration, the heavily involvement of the valve system would not make it possible for safe usage in the hospital as well as in homecare when the elders are the most populated users. The complicated system also makes it impossible for the mobility which is often the case for the oxygen therapy. The system is also incompatible to the existing oxygen delivery system that makes the usage practically difficult. The system also misses a fatal component that the medical doctors or the nurses who administrate the therapy are out of the control loop. For the feedback with the blood oxygen level, the disclosure by Schmidt et al. did not elaborate the precise control of the oxygen dosage. The rotameter used to meter the oxygen would not be able to handle the multiple valve system where the system pressure would vary, and the feedback is actually not feasible by valves only as it is known that the only measurement component in the system is the rotameter which is a pure mechanical component without direct signal output, and the reading will be easily altered with the system pressure changes. The system again is a complicated system without the capability of mobility as well as the missing links to the system administrators.

### SUMMARY OF THE INVENTION

It is therefore the objective of the present disclosure to provide the design of a apparatus and the corresponding system that shall be used to seamlessly replace the current mechanical oxygen therapy delivery with added features that shall allow both the therapy administrator and the patient to be beneficial from the disclosed system such that the oxygen therapy can be fully automated and efficient, and any cross-infections between the administrator and the patient from the mandatory close contact with the existing therapy system can be eliminated. The current inaccuracy of the oxygen metering with the mechanical rotameter can be replaced with the mass flow metering while the ultimate goal for oxygen assisted recovery of patient's blood oxygen level or concentration can be included into the disclosed apparatus and system forming the feedback loop for the realization of the automation. It is further desired that the instant data can be directly relay to the controlled data center and any alerts during the therapy can be relayed to the medical control center or to the destinated therapy administrator. The said device shall also be compatible and a direct replacement of the current pure mechanical oxygen therapy device without losing any advantageous features of the existing device, in particular of the portability. The device shall be stand-alone with the ability to be wirelessly interacting with the smart devices such as a smart phone in the case of homecare that are at reach by the users at any time. The disclosed apparatus and system can be further communicating with the destined cloud for data process or computing that relays to the destinated data control center.

In one preferred embodiment, the disclosed fully automated oxygen therapy apparatus shall have the said MEMS mass flow meter for metrology of the delivered oxygen and a valve to control the desired delivery via the feedback from the instant measurement of the blood oxygen level of the patient. The data during the therapy shall also be instantly relayed to the therapy administrator as well as to the patient via wireless transmission if desired. The said apparatus shall have the close proximity to the existing mechanical system such that it shall not require additional training to the practitioners, whilst only to facilitate and reduce the complicity of the current practice. The said apparatus shall be powered by battery as a stand-alone unit with the MEMS mass flow meter for direct measurement of the oxygen delivery to replace the mechanical rotameter while the mechanism to attach the water bottle for humidifying the oxygen gas before reaching to the patient shall be kept unchanged. The said MEMS mass flow meter shall directly and continuously measure the totalized oxygen delivered to the patient without the necessity for additional temperature and pressure measurement. The said MEMS mass flow meter shall display both the instant oxygen flowrate and the totalized oxygen consumed such that the status of the supply shall be more precisely registered as the said mass flow meter is far sensitive to the gas status as compared to the readings with the current mechanical rotameter which has no ability for the totalization. This is particularly important as in the situation of a pandemic as the COVID-19 when the oxygen delivery has to be done via an oxygen gas cylinder where the patients may be more than the numbers of hospital beds or direct oxygen lines available. The timely status of the gas consumption in the gas supply cylinder will allow the administrator to coordinate with the logistics and reduce the risk of insufficient oxygen supply in the middle of the therapy. The said apparatus shall have an adjustable valve that can provide and adjust the desired amount of the oxygen to the patient via the feedback from the instant measured blood oxygen level of the patient. A pre-registered value of the normal blood oxygen level shall enable the valve to cut off the oxygen supply when the patient is recovered, and the process can be relayed wirelessly to the therapy administrator as well as the patient without any manual intervening and attentions by the administrator during the whole therapy. The said MEMS mass flow meter further have the integrated line pressure sensor that shall be used together with the mass flow meter to alert the administrator if any abnormal oxygen supply occurred and minimize the risk of the ineffectiveness of the therapy.

In another preferred embodiment, the disclosed apparatus shall have the said MEMS mass flow meter with an adjustable valve as well as the instant blood oxygen level for the control of oxygen delivery, and the capability of wireless data relay to the oxygen therapy administrator as well as the patient with the fully automation close loop. The said MEMS mass flow meter is the key metrology device for metering the oxygen delivered to the patient replacing the mechanical rotameter. The meter shall meter the instant oxygen flowrate as well as the totalized oxygen delivered to the patient during the entire therapy time period. These values shall be wirelessly transmitted to the oxygen therapy administrator as well as to the patient and shall also be displayed locally with an LCD or LED or OLED display. In such an arrangement, the said MEMS mass flow meter shall have the same mechanical connections to the source of the oxygen supply, either a direct oxygen line from the hospital ward wall or an outlet of an oxygen gas cylinder. The complete flow channels and the corresponding sensing electronics shall be embedded inside the meter enclosure that shall be compatible with the current mechanical oxygen therapy devices. The outlet of the meter shall be connected to the water bottle for humidifying the oxygen gas before delivering to the patient. The base for the apparatus shall be made completely with biocompatible materials for oxygen delivery such as copper, stainless steel or biocompatible plastics. Both inlet and outlet of the said apparatus shall be made in the format of a female thread to adapt to various male adapters used in different areas or countries.

In another preferred embodiment, the disclosed apparatus shall have the said MEMS mass flow meter with an adjustable valve as well as the instant blood oxygen level for the control of oxygen delivery, and the capability of wireless data relay to the oxygen therapy administrator as well as the patient with the fully automation close loop. The said instant blood oxygen level measurement shall be achieved via the existing FDA approval oximeter with light-based infrared sensor clipped onto one of the patient fingers. The oximeter data are directly collected by the control electronics of the said oxygen therapy apparatus during the whole therapy process. The data interface shall be normally the standard I²C bus or other data transmission interface. The oxygen therapy administrator shall set either locally or remotely via the wireless data interface the desired recovery blood oxygen level based on the specific patient's conditions. This pre-set value shall allow the said apparatus to trigger the valve functions to either increase or decrease the supply as well as cut-off the supply when the desired blood oxygen level is recovered. In case of homecare, the apparatus shall offer the user menu for the selection of the target recovery blood oxygen level and the patient shall be able select the best value based on his/her therapy administrator's instruction. Further the administrator may also remotely monitor the instant process data from the patient who performs the therapy at home. This integration allows the therapy process to have the instant feedback to the oxygen flow to the patient without a manual intervention.

In another preferred embodiment, the disclosed apparatus shall have the said MEMS mass flow meter with an adjustable valve as well as the instant blood oxygen level for the control of oxygen delivery, and the capability of wireless data relay to the oxygen therapy administrator as well as the patient with the fully automation close loop. The said valve shall be preferably a proportional valve such as a solenoid valve which can adjust the amount of oxygen flow to the patient in accordance with the instantly measured blood oxygen level. The timely adjustment of the oxygen delivery shall ensure the needed oxygen dosage matches the recovery process of the patient's blood oxygen level and also shall avoid excessive dosage which is nontrivial for homecare applications as oxygen is normally supplied by the gas cylinders with limited oxygen mass or volume. In case of the therapy is programmed with a constant oxygen dosage, the valve can also be a constant open valve which only serves a cut-off of the supply at then end of the therapy to release the patient and preventing the excessive dosage. The MEMS mass flow meter, the instant oximeter and the automatically adjustable valve thus form a close loop for achieving the fully automation of the oxygen therapy.

In another preferred embodiment, the disclosed apparatus shall have the said MEMS mass flow meter with an adjustable valve as well as the instant blood oxygen level for the control of oxygen delivery, and the capability of wireless data relay to the oxygen therapy administrator as well as the patient with the fully automation close loop. The said apparatus shall have more than one embedded data storage that shall record the process of each of the completed oxygen therapy process in additional to the data relay via the wireless communication. The preservation of a local data is nontrivial as the pattern of the therapy shall be used for analysis the patient's blood oxygen level recovery, and with the machine learning algorithm, it can improve the subsequent therapy process and provides the ultimate benefits for the patient while the database shall also be used to assist the oxygen therapy administrator or the medical doctors to analysis the medication process. The local data storage shall also prevent any possible data loss due to the wireless data transmission process.

In yet another preferred embodiment, the disclosed apparatus shall have the said MEMS mass flow meter with an adjustable valve as well as the instant blood oxygen level for the control of oxygen delivery, and the capability of wireless data relay to the oxygen therapy administrator as well as the patient with the fully automation close loop. The said automated oxygen therapy apparatus shall further have the low energy version of wireless data communication. For applications in a hospital where multiple apparatus shall be used simultaneously, the preferred wireless option shall be LoRaWAN (long range wide-area network) which is a self-sustained network system provides both the data security and effectiveness of a local data transmission network without dependence on a third-party service provider. For the applications of homecare, the preferred wireless data shall be via a low energy Bluetooth communication components embedded inside the said apparatus. The Bluetooth module inside the apparatus shall be enabled to talk with a mobile device such as a smart phone that is widely available or accessible for the relevant users. The software designated to be run on the smart devices or the APP shall be used for data logger and/or analysis for the interactive information of the therapy process. These data registered in the said apparatus can also be downloaded to the smart devices via a data port such as a USB data ports in case the wireless communication has deficit. Either the APP or the data connection via the data port shall also allow the user to program the said apparatus such that additional functions such as time, blood oxygen level, gas flowrate, alarm or other parameters can be customized. Alternatively, for homecare applications, the wireless communication can be achieved with the NB-IoT (NarrowBand-Internet of Things) network, which can directly relay the data to the designated data center or the Cloud. The instant remote data shall allow the therapy administrator to remotely monitor the process, preventing the direct contact cross infections, and also efficiently manage the logistics of the oxygen supply in particular for the homecare applications. In an additional preferred arrangement, the smart device shall further relay the data to or receive the instructions from the designated data center or Cloud that have hosted the database for the therapy administration. In another preferred embodiment, the disclosed apparatus shall have the said MEMS mass flow meter with an adjustable valve as well as the instant blood oxygen level for the control of oxygen delivery, and the capability of wireless data relay to the oxygen therapy administrator as well as the patient with the fully automation close loop. The said apparatus shall be powered by battery for the often-needed requirement of portability, in particular when the oxygen supply is limited by the direct pipelines and gas cylinder supply is a must such as the case of a sudden increase of the patients in COVID-19 situation and homecare applications. Alternatively, a rechargeable battery cable as well as a wall power cable shall be included in the apparatus for backup.

The present disclosure provides a new design of an automatic yet compatible oxygen therapy apparatus where the mechanical rotameter is replaced with a MEMS mass flow meter that shall be capable of continuously and precisely metering the oxygen delivered, an integrated oximeter for instant measurement of the blood oxygen level, and a proportional valve to adjust the desired amount of the oxygen to the patient. The apparatus shall further relay the instant therapy process data to the user and further to the therapy administrator. These and other objectives of the present disclosure shall become readily apparent upon further review of the following drawings and specifications. Additionally, for those with the knowledge of the art, the regulated automatic oxygen therapy apparatus could be further utilized for gas delivery metering or dispensing via a fixed gas sources or a gas generator.

### BRIEF DESCRIPTIONS OF THE DRAWINGS

FIG. 1 is the prior art for a typical currently used oxygen therapy delivery device for both hospital and homecare applications.

FIG. 2 is the explosive view of the automated oxygen therapy apparatus disclosed for the replacement of the mechanical oxygen delivery for both hospital treatment and homecare applications. The said apparatus is a fully digitized mass flow meter device with integrated oximeter and adjustable valves and remote data transmission forming a close loop control system that shall be capable of providing an approach for assisting patient recovering from the loss of blood oxygen level via oxygen therapy without human intervention.

FIG. 3 is the said full assembled automated oxygen therapy control units with local display as well as the adjustable valve, data communication and local programmable access with the bottom oxygen outlet to the fixed liquid bottle for oxygen humidification before deliver to the patient.

FIG. 4 is the said full assembled automated oxygen therapy control units with local display as well as the adjustable valve, data communication and local programmable access with the side oxygen outlet to the disposable liquid bottle for oxygen humidification before deliver to the patient.

FIG. 5 is the said complete system of the full assembled automated oxygen therapy control units with local display as well as the adjustable valve, data communication and local programmable access, and the inclusion of the integration of the oximeter. The humidified oxygen outlet is located on the liquid bottle.

FIG. 6 is a schematic showing the operation flow chart including the component interactions, and functions of the said fully automated oxygen therapy system.

### DETAILED DESCRIPTION OF THE PREFERRED EMBODIMENTS

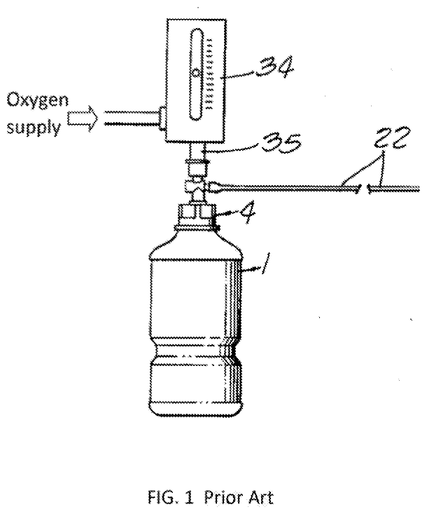

For a typical medical oxygen therapy delivery system is shown in the FIG. 1, which is a prior art (CJ. McPhee, Port system for medical humidifier container, U.S. Pat. No. 3,846,518, Nov. 5, 2974). In this current system, the oxygen will be supplied via a manually adjustable flow rate measurement rotameter (34), and then the flowrate regulated oxygen will pass via the bottom of the flowmeter (35) to the bottle with liquid (1) which is normally sterilized or medical compatible water. The bottle to the flowmeter is connected via the cap (4), and the oxygen will flow through the water to get humidified and then is delivered via the outlet (22) to the patient. Therefore, in order to initialize the oxygen therapy, an therapy administrator must be present on the scene to switch on the oxygen supply which is normally regulated with a mechanical pressure gauge, and then the administrator needs to adjust the flowrate according to the experiences as the rotameter has a very low accuracy and it is pressure sensitive. Hence the amount of the oxygen to be delivered to the patient would be often arbitrary. Since the therapy requires hours to be complete, it is not feasible for the administrator to be constantly attentive on the scene. In addition, the purpose of the oxygen therapy is to boost the patient's blood oxygen level which could also need to be manually acquired by the administrator. If the amount of oxygen delivered to the patient were not properly registered, there would be required for multiple manual adjustment with each additional measurement of the blood oxygen level. The whole therapy process is then labor intensive and the administrator might risk cross infections in case of the patient having a transmissive disease such as the COVID-19. These issues would be more problematic for the homecare therapy process. Therefore, the said disclosure shall address these and all the related items to provide a fully automated oxygen therapy apparatus.

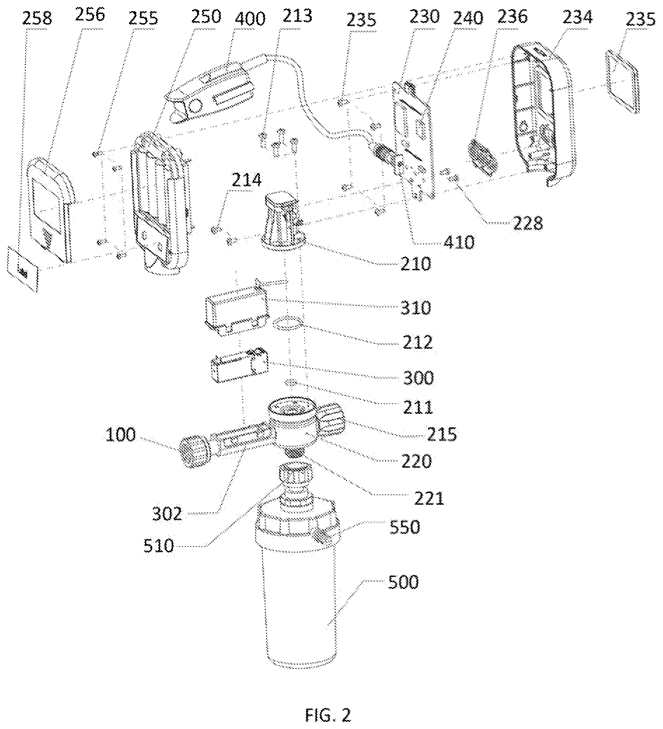

For the preferred embodiment, the present disclosure of a fully automatic oxygen therapy apparatus for boosting patient's blood oxygen level shall have a MEMS mass flow meter to replace the mechanical rotameter such that the amount of oxygen to be delivered to patient can be continuously and precisely metered. The integrated oximeter shall also be used to continuously monitor patient's blood oxygen level recovery, and the electrically adjustable valve shall be used to timely alter the desired amount of oxygen to be delivered to the patient based on the blood oxygen level recovery process. And the data during the whole therapy process shall be relayed to both the therapy administrator and the patient via wireless cloud data relay or via a smart device. The explosive view of the said fully automated oxygen therapy apparatus is shown in FIG. 2, where each component of the apparatus is disclosed. The oxygen is via the connector 100. The connector can be made of metals such as copper or stainless steel. It is screwed-in and is exchangeable as the connector specifications are different from country to country and even different within the same country but different locations. The oxygen supplied is normally pressurized but is only mechanically regulated with a set value. This value may change when the line pressure is various leading to the unstable of the amount of oxygen delivered to patient. The metrology unit 210 is fixed onto the oxygen dispensing body 220 with a set of gasket 211, 212 and four screws 213. The metrology unit 210 can be made of medical compatible metal such as copper or stainless steel or molded biocompatible plastics. It contains the preferred MEMS mass flow sensing elements installed in a top-down flow channel to re-direct the oxygen gas to the oxygen dispense body 220. The valve 300 is installed right after the oxygen supply connector 100 onto the inlet flow channel valve base (302) to the oxygen dispense body 220. The valve is preferably a proportional valve that functions to adjust the amount of oxygen to be delivered to the patient. This function is realized by the control electronics which simultaneously acquires the data from the oximeter (400) and the mass flow metrology unit (210). For a specific time period, if the patient's blood oxygen level recovery is not desirable, or the readings from the oximeter does not show the recovery progress, the control electronics will use the data from the oxygen mass flow metrology unit to control the proportional valve to alter the amount of the oxygen delivered to the patient for the optimized therapy process. The valve will be remaining constant open unless at the end of the therapy. For a simplified therapy process with a constant oxygen supply, the valve 300 will also be an ON/OFF constantly open valve which will then only serve for the cut-off the supply at the end of the therapy. 310 is the enclosure for the said valve to provide the protections of the valve operation.

The control unit of the said apparatus is composed of several components as shown in FIG. 2. 234 is the front cover of the enclosure made of hard plastics which hosts the control electronics, power supply and interfaces. 235 is the display protection that will be made of transparent light plastics or fiber glass. 236 is the user interface menu buttons made of hard plastics and its one face is embedded into the front cover 234 and another side is in direct contact with the control electronic printed circuitry board 230 with further secured with two screws 228. The four screws 235 are used to fix the control electronic printed circuitry board 230 to the front cover 234. 230 is the electronics on the printed circuitry board for the central control unit of the said automatic oxygen therapy apparatus, which contains the LCD or OLED display with the micro-processor unit (MCU) and signal conditioning circuitry for acquiring the oxygen mass flow data from the MEMS sensing elements using components such as analog to digital convertor (ADC), amplifiers and other necessary electronics components. The MCU also interfaces with the proportional valve 300 and the oximeter 400. The wireless communication chip 240 as well as the antenna are also part of the central electronic control printed circuitry board 230. For the said automatic oxygen therapy apparatus, the preferred wireless communication will be LoRa wide area network for the hospital applications as it provides the ultimate data safety and it is a self-supported local network without the requirements of the third-party service provider. However, for the homecare applications, the preferred wireless communication approach shall be NB-IoT (narrow band internet of things) which provides the direct data transmission to the designated cloud or data center. Alternatively, Bluetooth LE can also be an option as the personal smart devices are widely available and can be readily connected. The function of long-range data transmission to the designated cloud or data center can be fulfilled by the smart devices. In addition, at least two physical memory chips such as e-flash are also installed on this unit with direct access by the MCU for data storage and data safety. A physical data port in the form of USB-C or mini-USB or micro-USB is also included for access to the data on board in case the wireless data access is being disabled or not readily accessible. The interface 410 to the oximeter 400 is preferred to be a plug-in socket with additional cable secure fixture, as the oximeter is often required separate approval of the authorized body such as FDA (US Food and Drug Administration). The mass flow metrology unit 210 is connected to the printed circuitry board and secured by two screws 214. 250 is the back cover of the said apparatus control unit, which also contains the battery pack compartment. The back cover 250 is engaged with the from cover 234 with the fixture on both of these two parts made with the mold tooling, and it further secured with four screws 255. 256 is the battery pack chamber cover and 258 is the product information label that can be fixed to the battery pack chamber cover via glue or as pre-printed sticker. After the assembly of the control unit of the said apparatus, the unit is then fixed via the oxygen outlet 221 to the liquid bottle 500 which is used to humidify the oxygen before release to the patient via the connector 550. The liquid used is preferred to be sterilized water and can be heated to a temperature in close proximity to human temperature for the ultimate comfort during the complete oxygen therapy process.

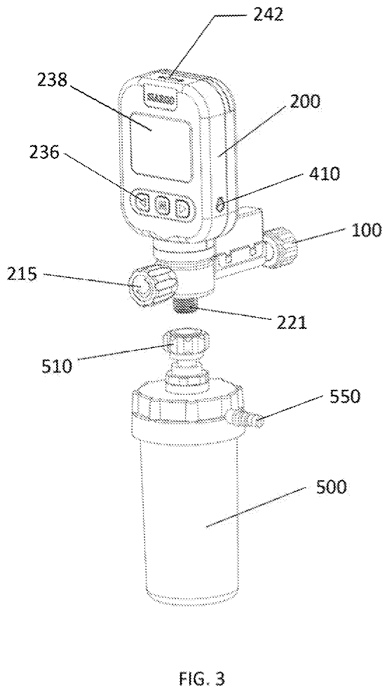

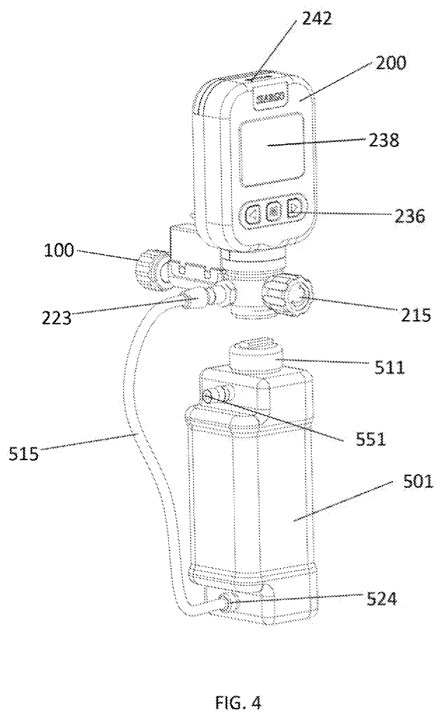

FIG. 3 exhibits the assembled fully automatic oxygen therapy apparatus with the fixed liquid bottle for oxygen humidification. The oxygen flown through the control unit 200 into the fixed liquid bottle that is engaged with the fixture 510 on the liquid bottle to the control unit outlet 221. In the preferred embodiment, the said apparatus shall be operated on battery pack that shall enable the continuous and precise measurement of the oxygen delivered while reference to the blood oxygen level acquired from the oximeter which is integrated via the port 410 into the control unit 200. The instant acquisition of the patient's blood oxygen level allows the control unit 200 to adjust the instant flowrate of oxygen via the proportional valve 300 such that the oxygen therapy can be optimized to the best interests of the patient. The manual valve 215 is kept for compliance purpose in case any failure of the proportional valve. It shall be normally kept at the full-open position. The keyboard with three functional keys 236 at the front face of the said apparatus control unit 200 are used to enter or program patient ID, desired oxygen therapy parameters including the therapy time, desired initial flowrate, alarms (such as battery status, flowrate, and blood oxygen concentration level), interval of data storage, designated wireless data option, reset the defaults as well as the desired recovery blood oxygen level. The access to these keys is normally password protected. These information are also instantly displayed on the local display 238 which is made of an LCD or OLED screen. The program functions can also be performed via the wireless data communications, which grants a better control to the therapy administrator especially for the homecare applications. The port 242 is a dual purpose one used for power backup purpose in case the battery failure and local data retrieval in case of wireless data communication errors. The port is preferably made of a Type-C USB connection or other easy to access USB port such as a micro- or mini-USB connector for easy access to the data stored on the local board. The threaded oxygen outlet 221 is compatible to the most of the current mechanical oxygen therapy approach where the oxygen will flow through the liquid or medical grade water inside the bottle and being humidified before delivering to the patient. In this configuration, the therapy administrator is often required to prepare the liquid bottle by manually filling the bottle with liquid such as sterilized or medical grade water. It is not only labor intensive but it risks for contamination, in particular for the homecare applications when the job is done by the patient or the caretaker who may not be well trained for this hygienic process. Therefore, in a preferred embodiment, a disposable liquid bottle with pre-filled medical grade liquid is used to address the concerns. This approach is also becoming more and more attractive in today's oxygen therapy process. For the preferred embodiment, in order to be compatible with the disposable liquid bottle attachment, the said oxygen outlet and the liquid bottle formality can be re-configured as shown in FIG. 4. The side outlet from the said apparatus is preferably made with the barbed connector 223 which can be directly connected with medical grade soft pipe 515 to the inlet of the disposable liquid bottle 524, and after oxygen flow through the liquid, it will be released humidified to be delivered to patient through the outlet 551. The engagement of the disposable liquid bottle 501 with the control unit 200 can be done with the engagement connector 511 that can be glided onto the bottle of the control unit 200. For the preferred embodiment, it is desired to have the humidified oxygen for patient at an elevated temperature above ambient and in close proximity to the human body temperature. Therefore, the said liquid bottle can also have a heater with a temperature sensor inside the liquid bottle for a feedback control for the liquid temperature. For the preferred embodiment, a temperature and humidity sensor can be installed to the liquid bottle outlet of 550 or 551 to allow the control unit to adjust the oxygen flow for the ultimate comfort of patient. Additionally the said liquid bottle will have a level sensor to detect the consumption of the liquid inside the liquid bottle and such a sensor can provide the warning of the low liquid level during the complete therapy process.

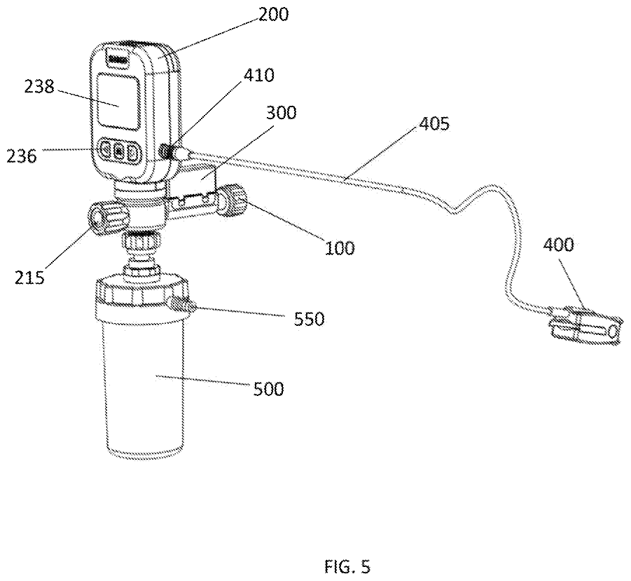

For the preferred embodiments, the said completed automatic oxygen therapy apparatus is exhibited in FIG. 5 where oximeter 400 will be a stand-alone unit but readily plug-in to the said automatic oxygen therapy control unit 200 via a medical grade cable 405. The oximeter is normally utilizing a transmission infrared sensor to meter the human blood oxygen level at the fingertip. The device is also normally required FDA approval for validated usage for medical purpose. While the oxygen therapy devices are normally considered to be Class I medical devices for its uncontrollable and non-repeatable process without mandatory metrology precision. The interface for the preferred embodiment is a medical grade socket which can tightly engaged to the said apparatus control unit 410. The data transmission is preferably using the I²C bus communication, and the power can also be supplied via the same port or independently supplied with a local battery inside the oximeter. In case that the oximeter is not connected, the unit shall be a complete and direct replacement of the current mechanical oxygen therapy apparatus with the automatic oxygen delivery. With the addition or integration of the independent oximeter unit, the oxygen delivery adds up the feedback loop and is directly correlated with the measure of the recovery of patient's blood oxygen level. The oxygen inlet 100 is an ISO-standard connector which is specific in different geographic regions. The proportional or ON/OFF oxygen compatible valve 300 is controlled by the control unit to deliver the desired oxygen flow via the liquid bottle 500 to have a humidified oxygen for patient. The desired therapy parameters can be entered via the local keyboard 236 and the information will be displayed instantly on the local display 238, or the data can be registered remotely via the wireless capability embedded inside the control unit 200. The said complete oxygen therapy system thus forms the said fully automatic oxygen therapy apparatus which shall not require any attention during the complete process of the oxygen and the remote data function enables the remote monitor of patient's recovery, which is especially helpful for the homecare applications.

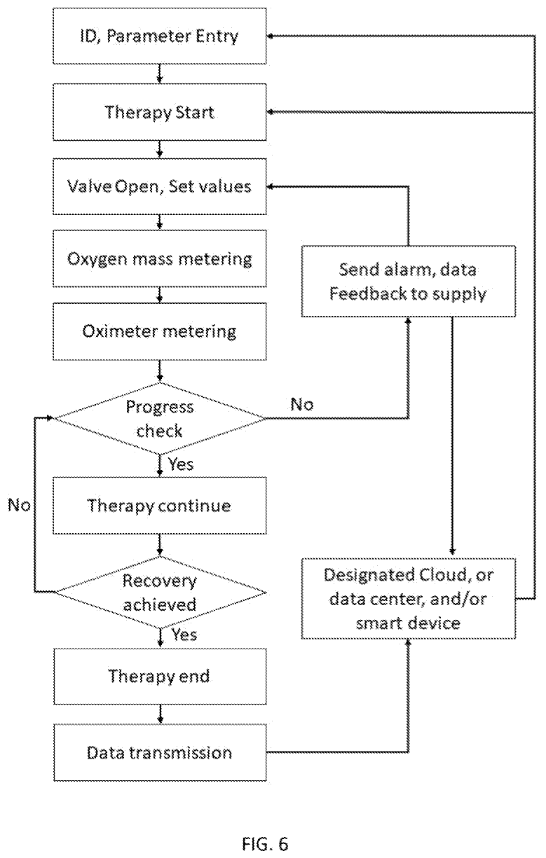

To further elaborate the advantages of the above preferred embodiments, FIG. 6 exhibits the operation flow of the said fully automatic oxygen therapy apparatus. In the preferred embodiment, the said process will first perform the “ID, Parameter Entry” by registering the patient identification, the desired oxygen therapy parameters such as, but not limited to the initial delivering oxygen flowrate, the targeted recovery blood oxygen level, initial checkup time frame, data transmission frequency, alarm for low oxygen supply, alarm for blood oxygen level, oxygen supply line pressure alarm level, and any other parameters that are relevant to the performance of the therapy. These parameters can be entered or changed via the keyboard on the control unit of the said apparatus and can also be entered via the connected smart devices or via the wireless data communications at the remote center by the therapy administrator. Once the desired setup is completed, or one can also use a pre-loaded setup parameters, the “Therapy Start” can be realized by one button push of the keyboard on the control unit, or the therapy can be initialized via the remote designated cloud or data center and/or smart devices at proximity. The said automatic oxygen therapy apparatus will then open the valve according to the set values to enable the oxygen flow to the patient. Simultaneously, the oxygen flowrate will be metered by the MEMS mass flow meter and the blood oxygen level of the patient will be measured by the connected oximeter. The control unit will start to see whether all pre-set values are properly executed, and if not, the feedback will adjust the amount of oxygen to be delivered, and the corresponding data and events will be transmitted to the designated cloud or data center. During the process, the said apparatus will also monitor other important events such as the liquid level inside the liquid bottle for humidification, and oxygen temperature and humidity values. Any deviations out of the pre-set value ranges, the said apparatus will trigger the warning and date transmission to the designated administrator. At the pre-set oxygen therapy time frame, the control unit will check the recovery of the blood oxygen level of the patient. If the recovery is achieved as desired, or before the set therapy time, the therapy will automatically end by cut off the oxygen supply and send the data to the designated cloud or data center, and/or to the designated smart devices. The patient will also be informed via the local control unit or the designated smart devices, and then will be released from the said therapy apparatus. If the desired recovery is not achieved at the pre-set time frame, the control unit will undergo the pre-set parameter check, and will transmit the data to the designated cloud or designated administrator or data center as well as designated smart devices. If the parameter verification is performed without issues, the therapy might be allowed to extend the therapy time frame for a pre-set extension and meanwhile such information will also be transmitted timely to the designated administrator or data center. The administrator will then have the authority to adjust the therapy or to determine the end of the therapy with additional options or immediately actions. Hence, the above described oxygen therapy process flow will not require any in person attention during the complete oxygen therapy process but allowing some remote intervention in case there are unexpected events. The automatic process will significantly relieve the intensive labor and presence of the administrator during the complete therapy process unless something unusual takes place for which the administrator can also be informed without any delay. This shall be very critical for the administrator for dealing with infectious diseases such as the COVID-19.

For the additional preferred embodiment, the said fully automatic oxygen therapy apparatus for those in the art shall become readily and apparently, and it could be further incorporated with additional features such as addition of an oxygen concentration sensor. It shall also be readily and apparently that the said cloud data can be directly interacting with the oxygen suppliers for the homecare applications to reduce the risk of insufficient therapy or completely out of supply. The said cloud data can also be further used for artificial intelligence data analysis for further improve the process flow of the oxygen therapy according to human age, pre-conditions, gender and other related parameters.

1. A fully automatic oxygen therapy apparatus having the MEMS mass flow meter to replace the existing mechanical rotameter and the integration of a proportional valve as well as an oximeter to fully control the delivery of the oxygen to patient with adjustment according to the feedback from the oximeter for human blood oxygen level. All process and data will be seamlessly relayed to the designated cloud, therapy administrator and/or data control center via wireless transmission while the apparatus will be fully granted the access to the designated and approved therapy administrator. The said apparatus is preferred to be powered by battery pack such that it can have the mobility to replace the current mechanical control unit in all applications. The said automatic oxygen therapy will be comprising:
A MEMS gas mass flow sensing unit that provides the precise, and temperature and pressure independent measurement for the instant flowrate as well as totalized oxygen delivered during the oxygen therapy;
An oximeter utilizing the transmission infrared sensing technology for measurement of the human blood oxygen level;
A proportional oxygen compatible valve that can adjust the supply of the oxygen within the therapy allowed range;
A local control unit with accessible keyboard that can enter the therapy parameters or recall the therapy recipe; store the therapy data; drive the proportional valve and acquire the instant data from the mass flow unit and oximeter;
A wireless data communication module for remote data register as well as remote access to the said apparatus;
A local display that relays the instant oxygen therapy process information, abnormal events as well assists the parameter register via the keyboard;
A physical data port via which the oxygen therapy data including the parameters and process data can be downloaded;
A liquid bottle that serves the purpose of adding humidity to the oxygen before delivery to the patient;
A battery pack with a backup wall plug power adapter and/or rechargeable power adapter;
A complete enclosure that houses the said components for being constituent into a complete and stand-alone automatic oxygen therapy apparatus Such an enclosure will also meet the safety requirements for medical applications.

2. The fully automatic oxygen therapy apparatus having the capability to precise metering and adjust the oxygen supply based on feedback from oximeter and to communicate with the remote designated administrator of claim 1 wherein the said MEMS gas mass flow sensing unit is able to provide the precision temperature and pressure independent measurement of the oxygen delivered during the complete therapy time frame. It is preferred that the measurement is utilizing the thermal mas flow or thermal time-of-flight sensing principle such that both the flowrate and pressure can be simultaneously measured. The full scale of the oxygen mass flowrate is preferred to be adjustable and covers both high and low flowrate oxygen therapy, and the preferred values are from 15 standard liter per minutes to 120 standard liter per minutes and to the maximal of 150 standard liter per minutes. The said mass flow sensing unit is also able to measure both the instant flowrate and totalized oxygen amount.

3. The fully automatic oxygen therapy apparatus having the capability to precise metering and adjust the oxygen supply based on feedback from oximeter and to communicate with the remote designated administrator of claim 1 wherein the said oximeter is preferred to be utilizing infrared transmission sensor to measure the human blood oxygen level. The oximeter is preferred to be a stand-alone unit that can be independently certified by a third party such as the Food and Drug Administration for the use of medical measurement. The oximeter is further preferred to have the digital output interface that can communicate with the control unit of the said automatic oxygen therapy apparatus.

4. The fully automatic oxygen therapy apparatus having the capability to precise metering and adjust the oxygen supply based on feedback from oximeter and to communicate with the remote designated administrator of claim 1 wherein the said control valve is preferred to be a proportional valve with the constant open option at power failure to ensure the delivery of oxygen to patient under power failure. The said proportional valve is driven by the control unit that determines the valve openness for the optimal oxygen flowrate to be delivered to the patient based on the individual therapy requirements. Alternatively, for the constant oxygen flowrate therapy, this said proportional valve can also be replaced with a constant open opted ON/OFF valve which will be used to cut off the oxygen supply at the end of the therapy for the automation therapy purpose.

5. The fully automatic oxygen therapy apparatus having the capability to precise metering and adjust the oxygen supply based on feedback from oximeter and to communicate with the remote designated administrator of claim 4 wherein the said flowrate control will also be able to be realized manually. The manual valve will be in service in case that the electrical power failure and the proportional or the ON/OFF valve is not functional such that the therapy can still be carried out and will not lead to any medical safety issue.

6. The fully automatic oxygen therapy apparatus having the capability to precise metering and adjust the oxygen supply based on feedback from oximeter and to communicate with the remote designated administrator of claim 1 wherein the said control unit will have the capability to have the oxygen therapy parameters as well as the patient identification or other related information or complete oxygen therapy recipe being manually entered into its data storage for process retrieval. The said control unit will also take the measurement data simultaneously from the MEMS mass flow sensing units and those form the oximeter to execute the user input therapy recipe for the optimal oxygen delivery to the patient. The said control unit will further have a plurality of numbers of memory chips or devices that allows the data can be simultaneously stored for data safety. It will also have other necessary functions such as allow the user to set password for protection.

7. The fully automatic oxygen therapy apparatus having the capability to precise metering and adjust the oxygen supply based on feedback from oximeter and to communicate with the remote designated administrator of claim 1 wherein the said wireless communication capability will be able to take the remote data entry as well as to transmit the data from the said control unit wirelessly to the designated cloud or administrator or data center or designated users. For the applications in hospital ward, the preferred wireless capability is low energy LoRa wide area network that is self-support and provides the required data safety and privacy. For the applications in homecare applications, the preferred wireless communication is low energy NB-IoT protocol such that the long-distance communication can be enabled. Alternatively, for homecare applications, the said control unit is preferred to be enabled by Bluetooth LE such that it is able to directly talk to a smart device that may provide a better wireless data transmission capability over NB-IoT due to the availability of the network. The said control unit will monitor the process parameters of the pre-entered or recalled recipe and make the oxygen delivery adjustment for the optimal results, and will also store locally and send remotely any alarms, abnormal event and the complete process data.

8. The fully automatic oxygen therapy apparatus having the capability to precise metering and adjust the oxygen supply based on feedback from oximeter and to communicate with the remote designated administrator of claim 1 wherein the said local display will be able to provide all the related information including instant oxygen flowrate, accumulated delivered oxygen, blood oxygen level, therapy time, battery or power status. Additional it will also display any alarm codes, liquid level and relative humidity of the oxygen delivered. For the control unit that has the stored therapy recipe, the display will also indicate the current recipe in use. The local display will also serve as the assistance to the use of keyboard for entering the patient information, parameters and or other related information.

9. The fully automatic oxygen therapy apparatus having the capability to precise metering and adjust the oxygen supply based on feedback from oximeter and to communicate with the remote designated administrator of claim 1 wherein the said physical data port via which the oxygen therapy data including the parameters and process data can be downloaded to a physical digital data processing devices such as a laptop computer. The physical data port on the said control unit will serve as a data backup in case of failure in wireless data transmission. The pre-arranged oxygen therapy recipes can also be uploaded to the control unit via this physical data port which is preferred to be made of a Type-C USB connection that is widely available to the general public. The connection cable can also be used for external power supply or battery recharge adapter cable. Alternatively, other commonly used data port formality can also be used such as but not limited to micro-USB, mini-USB, DB9, RJ connections.

10. The fully automatic oxygen therapy apparatus having the capability to precise metering and adjust the oxygen supply based on feedback from oximeter and to communicate with the remote designated administrator of claim 1 wherein the said the liquid bottle that serves the purpose of adding humidity to the oxygen before delivery to the patient. The said liquid bottle can be in two formality. A reusable one which is directly engaged via a mechanical connection to the control unit. Another option is a pre-filled disposable medical grade liquid bottle that is connected to the control unit via a soft medical compatible pipe. The preferred option will be the disposable one that is pre-filled with medical grade water such as sterilized water. For the preferred option of the disposable liquid bottle, a level sensor such as an ultrasonic sensor is installed at the bottom of the control unit where the liquid bottle is engaged. This sensor is preferred to transmit the data via a wired connection to the control unit once the liquid level is below 3 mm above the bottom, but most preferably below 5 mm above the bottom. It is also preferably such an alarm can be programmed depending on the therapy recipe such as oxygen delivered flowrate and time. Additionally, a humidity sensor will be installed at the exit of the oxygen on the said liquid bottle. This sensor is preferred to be a stand-alone in a capsule that can be directly engaged to the exit of the oxygen in the liquid bottle, and its data transmission is also realized via wired transmission mode to the control unit. The humidity sensor will monitor the water vapor concentration in the oxygen to be delivered to patient and feedback to the oxygen flowrate that can be adjustable via the control unit.

11. The fully automatic oxygen therapy apparatus having the capability to precise metering and adjust the oxygen supply based on feedback from oximeter and to communicate with the remote designated administrator of claim 10 wherein the liquid bottle can have the function of heating with a temperature control feedback. The heating capability will allow the humidified oxygen be kept at a temperature in proximity to the human body temperature, which will provide the ultimate comfort for patient dur the complete oxygen therapy process.

12. The fully automatic oxygen therapy apparatus having the capability to precise metering and adjust the oxygen supply based on feedback from oximeter and to communicate with the remote designated administrator of claim 1 wherein the said battery pack with a backup wall plug power adapter and/or rechargeable power adapter. The battery option will allow the unit to be capable of the ultimate mobility which is important for the homecare applications where the oxygen supply is mostly by the medical oxygen gas cylinder. The mobility capability is also critical for the cases when the visiting patients are overwhelming for the hospital capacity in the pandemic situation such as the COVID-19 when the patient will be treated with the oxygen therapy from oxygen gas cylinders.

13. The fully automatic oxygen therapy apparatus having the capability to precise metering and adjust the oxygen supply based on feedback from oximeter and to communicate with the remote designated administrator of claim 1 wherein the said the complete enclosure that houses the said components for being constituent into a complete and stand-alone automatic oxygen therapy apparatus. The enclosure will meet the safety requirements for medical applications. It will be made preferably with sturdy medical compatible engineering plastics or other specific materials for the requirements of the medical environments.
